(19) United States
(12) Patent Application Publication
(10) Pub. No.: US 2026/0140207 A1
(43) Pub. Date: May 21, 2026

(54) METHOD FOR CALCULATING A MULTI-DIMENSIONAL IMAGE-DOMAIN SYSTEM MATRIX FOR MPI MEASUREMENTS USING A MULTI-DIMENSIONAL SINOGRAM-BASED SYSTEM MATRIX

(71) Applicant: Bruker BioSpin GmbH & Co. KG, Ettlingen (DE)

(72) Inventors: Serhat ILBEY, Waldbronn (DE); Jochen FRANKE, Karlsruhe (DE)

(21) Appl. No.: 19/395,326

(22) Filed: Nov. 20, 2025

(30) Foreign Application Priority Data
Nov. 20, 2024 (EP) 24214298.2

(51) Int. Cl. G01R 33/12; A61B 5/0515; G01R 33/00

(52) U.S. Cl. CPC G01R 33/1276; G01R 33/0023; A61B 5/0515

(57) ABSTRACT

A magnetic particle imaging (MPI) measurement method includes generating a multi-dimensional image-domain system matrix A using an MPI-sequence with a field free line, using a multi-dimensional sinogram-based system matrix

$${A}_{Sinogram} ( t , r , z ) ∈ {ℝ}^{[ T , {N}_{r} , {N}_{z} ]} ,$$

comprising the following steps:
for each time point t_{c}, c∈[1, T]:
   get a time slice matrix A_{c}=A_{Sinogram}(t=t_{c}, r, z)∈ [symbol] ^{[N}^{ᵣ}^{,N}^{_{z}}^{]},
   replicate the time slice matrix A_{c} Nᵣ−1 times and copying these replications along a newly added 2^{nd} offset-dimension, resulting in a time volume matrix

$${A}_{c}^{′} ∈ {ℝ}^{[ {N}_{r} , {N}_{r} , {N}_{z} ]} ,$$

rotate the time volume matrix

$${A}_{c}^{′}$$

by the FFL θ_{c}=θ(t=t_{c}) at time point t=t_{c} in its 1^{st} and 2^{nd} offset-dimensions, resulting in a rotated matrix

$${A}_{c}^{{θ}_{c}}$$

concatenate the rotated matrices

$${A}_{c}^{{θ}_{c}}$$

of all time points along a time-dimension and vectorize the spatial dimensions (x, y, z), resulting in a constructed system matrix cSM∈ [symbol] ^{[T,(N}^{ᵣ}^{×N}^{ᵣ}^{×N}^{_{z}}^{)]}, and
perform a Fourier transformation on the constructed system matrix cSM on its time-dimension resulting in the multi-dimensional image-domain system matrix A.

### BACKGROUND OF THE INVENTION

### Field of the Invention

The invention concerns a method for calculating a multi-dimensional image-domain system matrix acquired from calibration measurements performed by an MPI Scanner using an MPI-sequence with a field free line FFL with FFL angles, FFL offsets r and FFL velocities.

### Description of the Related Art

In magnetic particle imaging (MPI) measurements, a spatially dependent magnetic field with a field-free region (e.g., a field-free point FFP or a field-free line FFL) is applied (selection field). FFL topology is in particular preferred for multidimensional imaging because it provides higher sensitivity than FFP topology, as all SPIONs across the entire FFL contribute to the signal (see, e.g., Weizenecker et al., “Magnetic particle imaging using a field free line,” Journal of Physics D: Applied Physics 41 (10): 105009, 2008). By applying a magnetic shift/focus field, the field-free region is moved through an examination volume (field of view FOV) along a trajectory with the aid of a measurement sequence in a drive-field region. The signal responses of magnetic particles, preferably superparamagnetic iron oxide particles (SPION), are measured as MPI signal data. The measured signal data are displayed as a sum signal of all excited particles. These are measured in the time domain and transformed into the frequency domain by a Fourier transform. A reconstruction is performed to produce the MPI image, e.g., using a system-function-based MPI image reconstruction.

In case of a system-function-based MPI image reconstruction, knowledge of a spatially encoded system response (frequency response), a so-called system function, is used, the latter describing the relationship between the measurement signal (MPI signal data) and, for example, the particle distribution of a certain particle class (mapping the particle concentration on a measured frequency response). As a rule, the system function is available as a “system matrix”, i.e., as discretized system function. The system matrix is provided for a system matrix region that comprises the part of the image domain where MPI image data are to be reconstructed. The system matrix describes the signal response of a calibration sample at various spatial positions. The system matrix is determined independently of the actual object measurement (e.g., by calibration measurement, by simulation, by hybrid approaches).

For the conventional system matrix acquisition, a system calibration sequence is performed repeating the MPI sequence to be performed and a calibration sample is placed at each voxel (3D pixel) position within the field of view (FOV). These calibration measurements are then used to construct the system matrix, designated with A^{M×N}, where M represents the number of the frequency components used, and N is the number of voxels. Finally, the image is reconstructed solving the ill-conditioned linear system of equations

$$A ρ = b$$

using iterative optimization methods, such as regularized Kaczmarz and FISTA, with the forward model

$${arg min}_{ρ} {‖ A ρ - b ‖}_{2}^{2} + λ Φ ( ρ )$$

where p is the image to be reconstructed, b is the measurement vector, Φ(·) is the regularization operator (for example, [symbol] ₁- and [symbol] ₂-norm of the image: ∥ρ∥₁ and ∥ρ∥₂, respectively), and λ is the regularization constant.

However, a fully-measurement-based calibration process can be impractical due to limited signal-to-noise ratio (SNR) and the need for a highly precise MPI-compatible 3D-axis-robot, which operates at limited positioning speed. As a result, acquiring a high-resolution system matrix can take several days depending on measurement time and the number of averages (see, e.g., Weizenecker et al., Three-dimensional real-time in vivo magnetic particle imaging”, Physics in Medicine & Biology 54(5):L1, 2009).

A classical system matrix calibration is disclosed in patent document US2015221103. This calibration method applies compressed sensing steps with a transformation matrix, which sparsifies the image reconstruction matrix, wherein the transformation matrix comprises analytic transformations.

Patent document US20170020407 describes an MPI method to determine calibration and measurement volumes, wherein the calibration volume is larger than the measurement volume and the overall measurement volume is arranged within the calibration volume (“overscanning”).

Kaethner et al., “Axially Elongated Field-Free Point Data Acquisition in Magnetic Particle Imaging,” IEEE Transactions on Medical Imaging, vol. 34, no. 2, pp. 381-387, February 2015 discloses a method for acquiring MPI data based on the axial elongation of a 2D FFP-trajectory. It is shown that such an elongation can be used as a data acquisition path to significantly increase the acquisition speed, with negligible loss of spatial resolution. However, as a result of the elongation, the 3D measurement volume is not sampled symmetrically and/or homogeneously which may lead to distortions and information loss in the reconstructed images.

Von Gladiss, A., et al., “Hybrid system calibration for multidimensional magnetic particle imaging,” Physics in Medicine & Biology 62.9 (2017) discloses the acquisition of a hybrid system matrix with a magnetic particle spectrometer. In contrast to changing the nanoparticle sample position, the nanoparticle sample stays at the same spatial position inside the sample chamber. Different spatial positions are emulated inside the magnetic particle spectrometer by changing the magnetic field that is applied to the particles. When acquiring a hybrid system matrix with a magnetic particle spectrometer the whole sample chamber represents one infinitesimal volume of the FOV in an MPI scanning device. Consequently, the calibration sample size is not constrained to the dimensions of a voxel of targeted image resolution, allowing for the use of a calibration sample as large as the sample chamber itself, provided the magnetic fields are homogeneous throughout, enabling high-SNR calibration measurements. The application of homogeneous offset fields generates the field sequence of this volume for all the particles inside the sample chamber. However, the magnetization response of SPIONs can be influenced by environmental factors such as temperature, and viscosity. Additionally, the non-deterministic field drifts and non-linearities present in the MPI scanner, etc. may not always be accurately emulated with a magnetic particle spectrometer and need to be compensated (e.g. transfer function compensation, background correction, sensitivity profile correction, . . . ).

Patent document CN 115797493 discloses a MPI sampling based on a FFL using an one-dimensional system matrix, where each projection angle (FFL angle) of the 2D imaging object is acquired separately in a step-wise manner. However, this method does not always work accurately and can only be used for a specific type of sequence.

U.S. Pat. No. 9,651,637 discloses a method for filtering out interferences from a received signal by applying signal-processing techniques like spectral cleaning.

Halkola et al., “System Calibration Unit for Magnetic Particle Imaging: Focus Field Based System Function”, Magnetic Particle Imaging, Springer Proceedings in Physics, vol. 140 discloses a method for generating a system matrix using an FFP. Instead of moving the nanoparticle sample position by a robot, magnetic focus fields are used for estimating the system function. This approach allows calibration measurements to be performed either using an external magnetic particle spectrometer (MPS) [Hybrid] or the MPI system itself without the need for additional hardware, thereby increasing the measurement duty cycle up to 100% by eliminating positioning time of the robot. Nevertheless, the full calibration procedure can still take several hours, during which the MPI system remains unavailable for imaging. Further, this method is not applicable for calibration measurements using FFL.

When using a field-free-line topology, the system function can be derived from a reduced dataset by leveraging the shift and rotation invariance of the MPI system (see, e.g., Mohn et al., “System Matrix Based Reconstruction for Pulsed Sequences in Magnetic Particle Imaging,” IEEE Transactions on Medical Imaging, Vol. 41, No. 7, July 2022, p. 1862-1873). For instance, the MPI signals at various FFL angles can be synthesized from a single-angle calibration measurement (see, e.g., Li et al., “Fast System Matrix Generation Based on Single Angle Calibration in Open-Sided Field Free Line Magnetic Particle Imaging,” IEEE Transactions on Biomedical Engineering, Vol. 71, No. 4, April 2024, p. 1209-1218). However, to accurately obtain the field-based system matrix applying constant (i.e., dc) focus field offsets, the FFL angle must remain fixed between repetitions.

### SUMMARY OF THE INVENTION

The present invention provides a method for lowering the total calibration time for generating a system matrix compared to conventional calibration and/or reducing mechanical effort required to position the source during calibration measurements. This is achieved by a method for generating a multi-dimensional image-domain system matrix as described herein.

According to the invention, a multi-dimensional sinogram-based system matrix A_{Sinogram}(t, r, z)∈ [symbol] ^{[T,N}^{ᵣ}^{,N}^{_{z}}^{]}, is used to generate the multi-dimensional image-domain system matrix A, where

$$r = offset in the offset - dimension of the sinogram$$

$$θ = angle in the angle - dimension of the sinogram$$

$$t = time in the time - dimension of the multi - dimensional sinogram - based system matrix {A}_{Sinogram}$$

$${N}_{r} = number of offsets$$

$${N}_{z} = number of voxels of a region of interest in a z - direction$$

$$T = number of time points of the time dimension ( number of calibration data points during an M P I imaging sequence )$$

The sinogram-based system matrix is acquired from calibration measurements using a field free line FFL with FFL angles, FFL offsets, and FFL velocities, the sinogram having an offset-dimension, an angle-dimension, and a z-dimension. The FFL is generated by means of selection fields and shifted by means of focus fields of an MPI scanner.

The inventive method comprises the following steps:

For each time point t_{c}, c∈[1,T]
get a time slice matrix A_{c}=A_{Sinogram}(t=t_{c}, r, z)∈ [symbol] ^{[N}^{ᵣ}^{,N}^{_{z}}^{]}, which comprises calibration data of the multi-dimensional sinogram-based system matrix A_{Sinogram}(t, r, z) at time point t=t_{c}, the time slice matrix having a 1st offset-dimension r and a z-dimension z,
replicate the time slice matrix A_{c} for each FFL-offset r, resulting in N, identical time slice matrices A_{c}, and copying these replications along a newly added 2nd offset-dimension, resulting in a time volume matrix

$${A}_{c}^{′} ∈ {ℝ}^{[ {N}_{r} , {N}_{r} , {N}_{z} ]} ,$$

and
rotate the time volume matrix

$${A}_{c}^{′}$$

by the FFL angle θ_{c}=θ(t=t_{c}) of the respective field free line FFL at time point t=t_{c} in its 1st and 2nd offset-dimensions, resulting in a rotated matrix

$${A}_{c}^{{θ}_{c}} ,$$

where the dimensions of

$${A}_{c}^{{θ}_{c}}$$

are associated with the spatial dimensions (x, y, z).

The rotated matrices

$${A}_{c}^{{θ}_{c}}$$

of all time points are concatenated along a time-dimension and the spatial dimensions (x, y, z) are vectorized, resulting in a constructed system matrix cSM∈ [symbol] ^{[T,(N}^{ᵣ}^{×N}^{ᵣ}^{×N}^{_{z}}^{)]}.

Finally, the multi-dimensional image-domain system matrix A is obtained after performing a Fourier transformation on the constructed system matrix cSM in its time-dimension.

The steps outlined above assume that the FFL rotation and FFL translation occur in the xy-plane, with a drive field (DF) defined along the z-axis. These steps should be modified as needed to accommodate different hardware configurations.

The replication of the time slice matrix is performed because it is assumed that the signals received from the spatial positions along the FFL are identical. This assumption best holds if the FFL is straight with zero field gradient along and at least as long as the scanner bore diameter.

At a given time point, the FFL has a specified FFL-angle. For rotating the time volume matrix, a rotation operator is applied to time volume matrix. Each slice z (slice of the time volume matrix having the same z-value) of the time volume matrix is rotated by the FFL-angle θ_{c} of the FFL at the respective time point. By rotating of the time volume matrix, the data of the calibration measurements is converted from sinogram domain to image domain using the FFL angle θ_{c} of the trajectory at the given time point.

For concatenating the rotated matrices, it is preferred to first create a matrix of zeros Aₜᵣₐⱼ∈ [symbol] ^{[T,N}^{ᵣ}^{,N}r^{,N}z^{]}. Then, the rotated matrices

$${A}_{c}^{{θ}_{c}}$$

are stacked into the matrix of zeros Aₜᵣₐⱼ along the first (time) dimension. By vectorizing the spatial dimensions of Aₜᵣₐⱼ, the constructed system matrix cSM is obtained.

The steps described above can be summarized as follows:

$$cS M = V \{ [ \begin{matrix}{A}_{1}^{{θ}_{1}} \\ {A}_{2}^{{θ}_{2}} \\ ⋮ \\ {A}_{T}^{{θ}_{T}}\end{matrix} ] \} = V \{ [ \begin{matrix}{ℛ}_{{θ}_{1}} \{ Ψ \{ {A}_{1} \} \} \\ {ℛ}_{{θ}_{2}} \{ Ψ \{ {A}_{2} \} \} \\ ⋮ \\ {ℛ}_{{θ}_{T}} \{ Ψ \{ {A}_{T} \} \}\end{matrix} ] \}$$

where Ψ is the operator replicating the 1st offset dimension of A_{c} of size Nᵣ×N_{z}, resulting in a Nᵣ×Nᵣ×N_{z} matrix, and V{·} is the operator, which vectorizes the spatial dimensions. By concatenating the rotated matrices, the conventional image-domain (robot-like) calibration data is constructed. In other words, in ideal conditions, i.e. where SNR is ultra-high, FFL is infinitely long, perfectly straight, and the fields of the MPI system are shift- and rotation invariant, the constructed system matrix cSM comprises data identical to the data that is acquired with a conventional robot-based calibration, where a calibration source is moved within the entire 3D field of view.

The multidimensional image-domain system matrix which should be used for reconstruction of MPI data is obtained by applying a Fourier transform to the constructed system matrix:

$$A = ℱ \{ cS M \}$$

where [symbol] is the Fourier transform operator along the time-dimension, and the resulting system matrix A is a K×N matrix, where K is the number of frequency components, and N=N_{xy}×N_{xy}×N_{z} is the total number of voxels of the calibration volume, assuming Nᵣ=N_{xy}.

In case there is no shift and rotation invariance, correction fields have to be determined.

The calibration signals of the calibration measurements are detected by one or more receiver coils. If multiple receiver coils are used, A must be calculated for each coil separately.

The multi-dimensional sinogram-based system matrix A_{Sinogram} can be obtained in different ways:

In a first variant (trajectory offset variant), the multi-dimensional sinogram-based system matrix A_{Sinogram}(t, r, z)∈ [symbol] ^{[T,N}^{ᵣ}^{,N}^{_{z}}^{]} is generated according to the following method, wherein the offset Δr is an FFL trajectory offset, i.e. a shift of the complete trajectory:

Prior to calibration measurements, the following is determined:
target positions for a calibration source,
a calibration volume in sinogram domain from which calibration data is to be acquired, the calibration volume comprising the region of interest and optionally an overscanning region (the region of interest is the region to be imaged; the overscanning region is the area of the calibration volume that is not to be imaged, but is used for calibration measurements),
MPI imaging sequence with a trajectory comprising sequence parameters, the sequence parameters including FFL angular frequency, FFL translational frequency (rate of the change of the translational velocity during a trajectory run), and MPI imaging sequence duration,
Tracer and/or environmental parameters,
FFL-trajectory-offsets Δr, by which the positions of the field free line during the trajectory are to be shifted within the calibration volume, perpendicular to the respective orientation of the field free line.

The calibration measurements are performed by running the MPI imaging sequence/trajectory at least once during each calibration measurement using an MPI scanner, wherein for each combination of FFL-trajectory-offset Δr and target position z a calibration measurement is performed.

The multi-dimensional sinogram-based system matrix A_{Sinogram} is determined from the calibration data.

The calibration data are acquired by applying a drive field to a calibration source (DF-excitation) and detecting the signal response.

The determined multi-dimensional sinogram-based system matrix is valid for specific tracer and/or environmental parameters. In case the tracer and/or environmental parameters change, a new multi-dimensional sinogram-based system matrix has to be determined.

Instead of a conventional calibration, which is done by repeating the MPI sequence (e.g., with the sinusoidal trajectory) by positioning a point-like source object at every point in the imaging volume, the MPI sequence is repeated for varying FFL trajectory offsets in sinogram domain, i.e., the FFR trajectory offset is applied to the entire trajectory, and each position of the FFL during the trajectory is shifted perpendicular to the orientation of the FFL at the specific position. The FFL velocities and FFL angles vary dynamically. After each calibration measurement (comprising at least one trajectory run) the FFL trajectory offset is changed. In contrast to conventional calibration, MPI sequence repetitions preferably comprise stepped target positions z, and FFL trajectory offsets that are not along the FFL-directions, since it is assumed that the signals received from the spatial positions along the FFL are identical. Thus, the number of calibration measurements can be significantly reduced using this dimensionality reduction.

In some variants the number of offset Nᵣ can be chosen smaller than the number of calibration voxels in the xy-plane, Nᵣ≤N_{xy} (if overscanning is ignored).

In some variants the number of offset Nᵣ can be chosen larger than the number of calibration voxels in the xy-plane, Nᵣ>N_{xy}.

Preferably, the target positions of the calibration source only vary in drive field direction. Drive field(s) are orthogonal to the focus fields driving the FFL angular frequency and FFL translational frequency. For example, FFL rotation and FFL translation are defined in the xy-plane and the drive field is defined in the z-dimension. In a special embodiment, there can be multiple drive field and focus field directions and drive fields and focus fields can be in the same dimension (superposition).

Since the invention is focused on system matrices for reconstruction of MPI data acquired using FFL, the trajectory is defined in the sinogram domain (r, θ). Accordingly, the calibration volume is defined in sinogram domain.

During each calibration measurement, the entire trajectory is run at least once, wherein for different calibration measurements the trajectory is run with different offsets. Averaging can be applied. If no averaging is applied, one calibration measurement comprises one full trajectory. If averaging is applied, one calibration measurement comprises a plurality of trajectory runs. Each calibration measurement results in one vector of data points.

Without averaging, the trajectory of the MPI sequence is run through N_{z} times for each FFL-trajectory-offset Δr and Nᵣ times for each target position z for the calibration source; in case of averaging, a multiple of the aforementioned numbers of trajectory runs is performed.

For determining the multi-dimensional sinogram-based system matrix from the calibration data, calibration data is acquired for various combinations of FFL-offset and source positions, each calibration data having T time points. The resulting data T×Nr×Nz form the multi-dimensional sinogram-based system matrix A_{Sinogram}.

Since the acquired calibration data originates from MPI hardware components such as power amplifiers, filters, etc., interferences with MPI receive coils can occur. In order to remove such interferences, signal-processing techniques like spectral cleaning can be applied to all calibration data and imaging data.

Preferably performing the calibration measurements comprises the following steps:
i. The calibration source is positioned within the calibration volume at a first target position,
ii. Calibration data is acquired with the calibration source being positioned at a first target position are performed, wherein the trajectory of the MPI sequence is run through until all FFL-trajectory-offsets Δr have been set, wherein the FFL-trajectory-offset Δr is changed after the trajectory is run through.
iii. The calibration source is positioned within the region of interest at a further target position that is shifted relative to the previous target position within the calibration volume in at least one direction, preferably along a drive field direction.
iv. The trajectory of the MPI sequence is run through until all FFL-trajectory-offsets Δr have been set, wherein the FFL-trajectory-offset Δr is changed after the trajectory is run through with the calibration source being positioned at the further target position,
v. Steps iii)-iv) are repeated until measurements for all target positions of the calibration source are performed.

In order to remove background noise, background measurements can be performed, preferably prior to positioning the calibration source at the target positions in step i and step iii, without the calibration source being in the calibration volume and/or with the calibration source being in a magnetically saturated state.

In a second variant (MPS variant), the multi-dimensional sinogram-based system matrix A_{Sinogram}(t, r, z)∈ [symbol] ^{[T,N}^{ᵣ}^{,N}^{_{z}}^{]} is generated according to the following method using a Magnetic Particle Spectrometer for emulating a calibration volume the method comprising:

Prior to calibration measurements, determine:
calibration volume in sinogram domain, from which calibration data is to be acquired, the calibration volume comprising the region of interest,
MPI scanner parameters of the MPI scanner including drive field frequency, drive field amplitude, and magnetic field gradient,
MPI imaging sequence with a trajectory comprising sequence parameters, the sequence parameters including FFL angular frequency, FFL translational frequency, and MPI imaging sequence duration,
FFL-trajectory-offsets Δr, by which the MPI imaging sequence is to be shifted within the calibration volume,
spatial positions z of the calibration volume,
tracer and/or environmental parameters,
for each combination of FFL-trajectory-offset Δr, and spatial position z: time dependent magnetic fields B(t, Δr, z), wherein each magnetic field B(t, Δr, z) corresponds to the magnetic field at generated by the MPI scanner during a trajectory run with a specific FFL trajectory offset Δr at a specific spatial position z,
   Calibration measurements are performed by emulating the calibration volume by applying the determined magnetic fields to a calibration source, wherein for each combination of FFL-trajectory-offset Δr and spatial position z a calibration measurement is performed, during which the corresponding magnetic field (t, Δr, z) is applied at least once, and calibration data are acquired,
   The multi-dimensional sinogram-based system matrix A_{Sinogram} is determined from the calibration data.

The calibration data are acquired by applying a drive field to a calibration source (DF-excitation) and detecting the signal response.

The determined multi-dimensional sinogram-based system matrix is valid for specific tracer and/or environmental parameters. In case the tracer and/or environmental parameters change, a new multi-dimensional sinogram-based system matrix has to be determined.

In contrast to the aforementioned variant, the calibration volume (i.e. the magnetic fields at different positions within the FOV of the MPI scanner) is emulated by an external Magnetic Particle Spectroscope. To emulate a calibration volume means emulating the fields generated by an MPI scanner during an MPI sequence for each repetition (Nᵣ×N_{z}).

The calibration source is positioned in a sample chamber of the MPS. By applying the emulated fields the calibration source experiences the same magnetic field as it would be positioned at a specific z-position in an MPI scanner while the trajectory is run with a specific trajectory offset (emulated calibration volume).

In a third variant (generic variant) the multi-dimensional sinogram-based system matrix A_{Sinogram}(t, r, z)∈ [symbol] ^{[T,N}^{ᵣ}^{,N}^{_{z}}^{]} is generated according to the following method, wherein the offset r is an FFL-offset r:
perform generic MPI calibration measurements by applying at least one drive field cycle of a drive field in each calibration measurement using an MPI scanner, wherein during each generic MPI calibration measurement data is acquired for a specific combination of values of target position for a calibration source, FFL offset, and of predetermined parameters, comprising FFL angle and FFL velocities,
determine a generic system matrix A_{generic}, where A_{generic} comprises calibration data acquired from the generic MPI calibration measurements, and
   From the generic system matrix A_{generic}, trajectory-specific calibration data is selected according to the following method:
for each drive field cycle of an MPI measurement
   determine the FFL angle, FFL offset, and FFL velocity,
   pick the calibration data from the generic system matrix A_{generic} of that calibration measurement that best matches to the combination of values of the predetermined parameters of the respective drive field cycle, and
   concatenate all picked calibration data along the time dimension, resulting in the multi-dimensional sinogram-based system matrix A_{Sinogram}.

Instead of trajectory-specific calibration measurements, a generic set of measurements (i.e., independent of any trajectory for a multitude of parameter value combinations) are performed so that the calibration data can be used for a multitude, in particular for all possible MPI sequences, i.e., such that a generic system matrix is generated. The dimensions of the generic system matrix correspond to the parameters, which are varied for the different MPI calibration measurements.

Prior to the generic MPI calibration measurements, the following is determined:
target positions for a calibration source,
parameters, wherein the parameters include sequence parameters such as FFL velocities of a field free line FFL of a magnetic selection field, and may comprise further parameters
FFL-offsets, by which the field free line FFL is to be shifted within the calibration volume,
   In variant 2 and variant 3, the number of offset N, is typically equal to the number of calibration voxels in the xy-plane, Nᵣ=N_{xy}. In case of overscanning and/or oversampling, Nᵣ>N_{xy}.

In a special variant, the parameters are discretized, thereby generating intervals of discretized parameter values for each parameter. The generic system matrix is then determined by determining individual acquisition system matrices A_{c}, wherein each individual acquisition system matrix A_{c} combines calibration data derived from different calibration measurements that are assigned to the same parameter value combination but different FFL-offsets, and combining individual acquisition system matrices A_{c} of different parameter value combinations. The MPI-sequence is divided into trajectory-sections, whereby the duration of each trajectory-section corresponds to the duration of a drive field cycle, each trajectory-section being associated with an actual parameter value combination, wherein during each trajectory-section a data-subset of the MPI-data is acquired. For each trajectory-section, the best matching individual acquisition system matrix A_{c} is picked from the multi-dimensional generic system matrix A_{generic}, wherein the best matching individual acquisition system matrix A_{c} is the one with the parameter value combination that best matches the actual parameter value combination of the respective trajectory-section, where [symbol] ∈[1, C] is the trajectory-section number and C is the total number of trajectory-sections. In this variant, calibration measurements are not performed by running trajectories, but by shifting the FFL to different offset-position, wherein for each offset position calibration measurements for a multitude of parameter value combinations are performed.

Preferably, the determined parameters comprise at least one of the following: particle type of used tracer of the calibration source, temperature of the calibration source, viscosity of the calibration source, velocity of the calibration source per acquisition window, DF frequency, DF amplitude, magnetic field gradient of an MPI system used for the MPI measurements, duration of the acquisition window, position of optional hardware inserts, shape of the field-free line. Although the consideration of additional parameters increases the total acquisition time and memory requirements, it enables the use of the same multidimensional system matrix for any arbitrary trajectory in a multitude of framework conditions. In this way, the MPI system user does not need to do very tedious calibration measurements repeatedly for each further parameter.

Alternatively, to the variant described so far, the multi-dimensional sinogram-based system matrix can be synthesized using model-based and/or hybrid MPI calibration techniques.

In a preferred variant, for setting a specific FFL-trajectory-offset the MPI sequence (first variant), or for setting a specific FFL offset (third variant) the field free line, is shifted in the offset-dimension of the sinogram by applying additional focus fields. The FFL-trajectory-offset or the FFL offset respectively can exceed half of the field of view to enable overscanning.

In case of limitations generating FFL-offsets due to, for example, limited peak power of power amplifiers, bore size, and overheating of hardware etc., the data can only be obtained for a limited range of field offsets, wherein “field offset” can be composed of the FFL-offset, the FFL-trajectory offset a z-offset or a combination thereof. The multi-dimensional sinogram-based system matrix A_{Sinogram} becomes, therefore, clipped in at least one of the spatial dimensions (r, z) of the multi-dimensional sinogram-based system matrix A_{Sinogram}, such that the multi-dimensional sinogram-based system matrix A_{Sinogram}, only comprises calibration data from field offsets lower than a threshold rₗᵢₘ, zₗᵢₘ.

If calibration data cannot be acquired for positions where r>rₗᵢₘ and/or z>zₗᵢₘ, these data gaps should be estimated using interpolation, extrapolation, or advanced techniques such as artificial intelligence, where rₗᵢₘ=Maximum FFL-offset in the xy-plane, for example the MPI system bore radius and zₗᵢₘ=Maximum FFL-offset or position offset of the calibration source along the z-axis.

In a preferred variant, during each calibration measurement, averaging is applied; preferably, the number of averages is set for a targeted signal-to-noise-ratio SNR. Averaging means repetition of a measurement with identical parameters. Averaging is used to decrease the level of noise floor. In case of the trajectory specific system matrix as described above, the entire trajectory is repeated during a calibration measurement. In the case of the generic system matrix, short trajectory sections are repeated during a calibration measurement. In case SNR of the signal of a calibration measurement is not sufficient, the required number of averaging can be determined experimentally for a targeted SNR. SNR of a measured signal is ideally proportional to the square root of the number of averages applied, if the noise is random, uncorrelated, and stationary.

In a highly preferred variant, the calibration source is moved relative to the region of interest exclusively in the drive field direction, in order to move the calibration source between the target positions.

In a preferred variant, a fully sampled calibration data consists of n measurements, the number of calibration measurements m, is smaller than the number n, and calibration data not measured is estimated, in particular by interpolation and/or extrapolation using the already acquired calibration data (undersampling). Due to geometrical symmetries, calibration data of some of the trajectory offsets can be generated using acquired calibration measurements, e.g. by phase shifting, amplitude scaling, and/or flipping.

For image reconstruction of MPI-data which have been acquired by performing an MPI sequence, a calculated multi-dimensional image-domain system matrix A is used, wherein a convex optimization algorithm is used which is based on a forward model, in particular the following:

$${arg min}_{ρ} {‖ {A}_{ρ} - b ‖}_{2}^{2} + λ Φ ( ρ )$$

$$\begin{matrix}where \\ A = calibration matrix , i . e . multi - dimensional image - domain system matrix , \\ ρ = M P I - image \\ b = measurement vector , \\ λ = regularization constant for regularization operation \\ Φ ( \cdot ) = regularization operator .\end{matrix}$$

When multiple receiver coils are used, the calibration matrices of each coil and measurement vectors of each coil can be merged (concatenated) prior to reconstruction. In other words:

$$[ \begin{matrix}A of receiver 1 \\ A of receiver 2 \\ ⋮ \\ A of receiver C\end{matrix} ] \to A and [ \begin{matrix}b of receiver 1 \\ b of receiver 2 \\ ⋮ \\ b of receiver C\end{matrix} ] \to b$$

where C is the number of receiver coils.

To improve the signal-to-noise ratio and/or image reconstruction accuracy, noisy frequency components can be removed from the multidimensional system matrices of the receiver coils and from the measurement vectors of the receiver coils. In this case, the multi-dimensional system matrices of the receiver coil and the measurement vectors of the receiver coils are reduced.

In a preferred variant, projections of MPI-image on the xz- or yz-plane at a specific angle θ_{projection} can be imaged by setting FFL angle θₜᵣₐⱼ(t)=θ_{projection} (a special variant of the sinusoidal trajectory). The result of image reconstruction, the MPI-image ρ, will be directly the projection of the MPI-image at the specific angle θ_{projection} without further post-processing.

In a preferred variant, (maximum intensity) projection of MPI image on any plane can be calculated by picking the maximal value or summing all slices of the reconstructed MPI-image p along the selected plane after reconstruction.

Further advantages of the invention can be derived from the description and the drawings. Also, the above-mentioned and the still further described features can be used according to the invention individually or in any combination. The embodiments shown and described are not to be understood as a conclusive list, but rather have an exemplary character for the description of the invention.

### BRIEF DESCRIPTION OF THE DRAWINGS

FIG. 1 shows the basic method steps of the inventive method for generating a multi-dimensional image-domain system matrix using a multi-dimensional sinogram-based system matrix.

FIG. 2 shows the steps of a first variant of generating the multi-dimensional sinogram-based system matrix using a trajectory specific calibration data acquisition by offsetting the trajectory.

FIG. 3a shows the temporal course of FFL offset (r) of a 3D FFL sinusoidal trajectory.

FIG. 3b shows the temporal course of FFL offset (r) of a 3D FFL sinusoidal trajectory with trajectory offset Δr.

FIG. 3c shows the temporal course of FFL-angle, which does not depend on trajectory offset Δr, during the sinusoidal trajectory.

FIG. 4 shows the steps of a calibration data acquisition with varying FFL-offset for each source target position for the first variant.

FIG. 5 shows the steps of a second variant of generating the multi-dimensional sinogram-based system matrix using a Magnetic Particle Spectrometer.

FIG. 6 shows the steps of a third variant of generating the multi-dimensional sinogram-based system matrix using a generic system matrix.

FIG. 7 shows a calibration volume with overscanning region with a superimposed trajectory.

FIG. 8a shows positions of the calibration source used for calibration with 1D movement of the calibration source.

FIG. 8b shows offset-positions of the FFL for the case that an FFL with extension in y-direction for FFL-angle is 0°.

FIG. 8c shows offset-positions of the FFL for the case that an FFL with extension in x-direction for FFL-angle is 90°.

FIG. 9 shows a region of interest and an FFL with its FFL-parameters.

FIG. 10a shows the temporal course of a 3D FFL sinusoidal trajectory with 1D excitation.

FIG. 10b shows the temporal course of the FFL-angle during the sinusoidal trajectory.

FIG. 10c shows the sinusoidal trajectory of FIG. 10a in a sinogram.

FIG. 11a shows a sinusoidal trajectory in sinogram-domain and image-domain.

FIG. 11b shows an offset sinusoidal trajectory in sinogram-domain and image-domain for a trajectory offset Δr=−¼ FOV.

FIG. 11c shows an offset sinusoidal trajectory in sinogram-domain and image-domain for a trajectory offset Δr=½ FOV.

FIG. 11d shows the offset sinusoidal trajectory of FIG. 11c clipped in sinogram-domain and image-domain.

### DETAILED DESCRIPTION

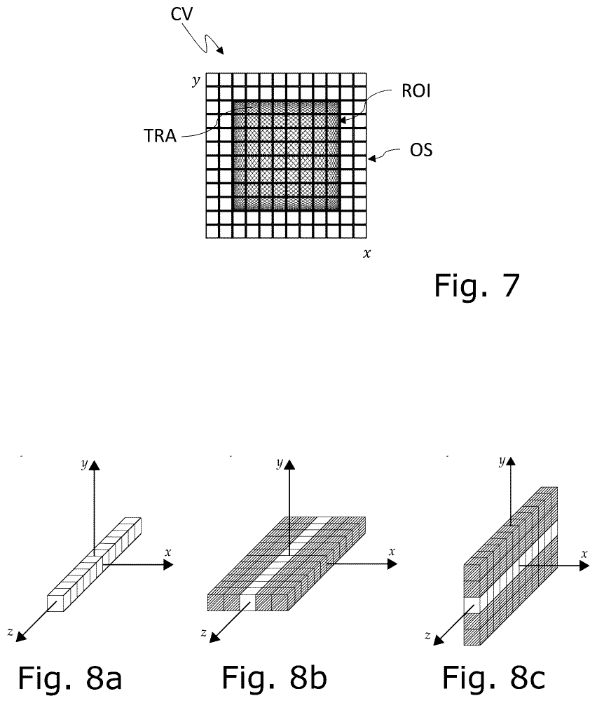

FIG. 7 shows a calibration volume CV (image domain x, y) with a region of interest ROI, from which an MPI-image is to be generated, and an overscanning region OS (2D-illustration). In a 3D calibration volume CV, a third dimension z is added (not shown).

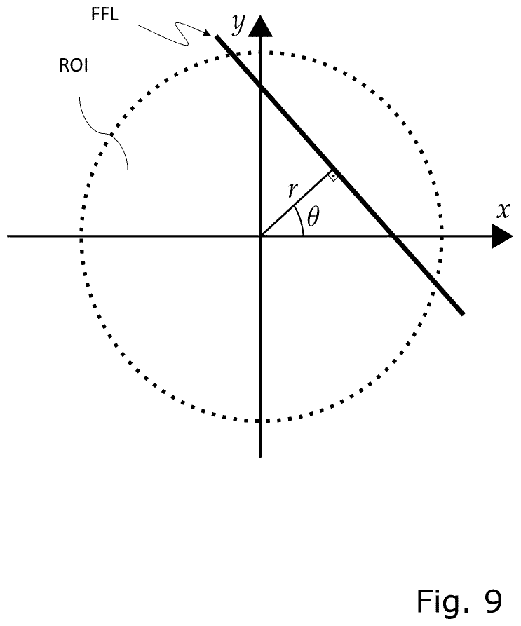

Within the region of interest, ROI a trajectory TRA is indicated as example along which a field free line FFL (see FIG. 9) is moved during applying an MPI-sequence. According to the prior art, the trajectory TRA is repeated for any calibration source position within the region of interest ROI. During tracing the trajectory TRA the FFL-angle, FFL-offset, FFL-angular-velocity, FFL-translational-velocity might change.

When using an FFL, the reconstruction cannot be performed directly to the image domain (x, y, z), but to a sinogram domain (θ, r, z):

FIG. 9 shows a field free line FFL in the region of interest ROI with an FFL-angle θ (angle between the FFL-orientation and the x-axis) and an FFL-offset r (distance of the FFL to the origin). During running a trajectory, the FFL is moved through the calibration volume and thus having different FFL-offsets during a trajectory run. Translation and rotation of the FFL take place within the x- and y-direction, which is defined by magnetic shift/focus fields and/or mechanical rotation. A 3D field of view FOV can be imaged by rotating and shifting the FFL in the x- and y-directions between drive field excitation cycles (step-wise) or during drive field excitation cycles (dynamically). In other words, by modulating the shift or focus fields to cover each (x,y) position, the field of view FOV can be traversed, for example, using a step-wise meander or dynamic sinusoidal trajectory, as shown in FIG. 10 for the dynamic case. A translation (offset) of the FFL corresponds to moving vertically in the 2D sinogram, whereas a rotation of the FFL corresponds to moving horizontally. Moving in the z-direction (e.g., by DF excitation or focus/shift fields) corresponds to moving orthogonal to the 2D sinogram.

The fields in 3D generated with the exemplary dynamic sinusoidal trajectory with 1D excitation in the z-direction can be defined with the following equations:

$$H ( θ ) = G [ \begin{matrix}\frac{1}{2} + \frac{1}{2} cos 2 θ & \frac{1}{2} sin 2 θ & 0 \\ \frac{1}{2} sin 2 θ & \frac{1}{2} - \frac{1}{2} cos 2 θ & 0 \\ 0 & 0 & 1\end{matrix} ] [ \begin{matrix}x \\ y \\ z\end{matrix} ] , θ = 2 π {f}_{rot} t$$

$${H}_{DF} ( t ) = [ \begin{matrix}0 \\ 0 \\ {A}_{DF} sin ( 2 π {f}_{DF} t )\end{matrix} ]$$

$${H}_{FF} ( t ) = [ \begin{matrix}{H}_{FF} sin ( 2 π {f}_{tra} t ) cos ( 2 π {f}_{rot} t ) \\ {H}_{FF} sin ( 2 π {f}_{tra} t ) sin ( 2 π {f}_{rot} t ) \\ 0\end{matrix} ] , {H}_{FF} = G \frac{F O {V}_{xy}}{2} ,$$

where G is the gradient of the selection field, A_{DF} and f_{DF} are drive field amplitude and frequency, respectively, and fᵣₒₜ and fₜᵣₐ are the rotational and translational frequencies of the trajectory, respectively.

FIG. 10 a-c show the temporal course of a 3D FFL sinusoidal trajectory (FIG. 10a), the temporal course of the FFL-angle during the sinusoidal trajectory (FIG. 10b) and a sinogram representation of the sinusoidal trajectory (FIG. 10c), each with 1D DF-excitation orthogonal to the shown plane.

By using 1D excitation in a z-direction, a 3D FOV can be imaged by rotating and translating the FFL in the x- and y-directions. This can be achieved, for example, with a sinusoidal trajectory as shown in FIG. 10, which can be described by the following equations:

$${r}_{traj} ( t ) = \frac{F O {V}_{xy}}{2} sin ( 2 π {f}_{tra} t )$$

$${θ}_{traj} ( t ) = 2 π {f}_{rot} t$$

where FOV_{xy} is the FOV in the xy-plane, t∈[0 T_{acq}], T_{acq} is the sequence duration, and fₜᵣₐ and fᵣₒₜ are the translational and rotational frequencies of the FFL, respectively. To ensure periodicity and the densest coverage in the sinogram representation of the trajectory shown in FIG. 10c for a given T_{acq}, fₜᵣₐ and fᵣₒₜ must be appropriately set.

According to the invention, a method is suggested which uses a multi-dimensional sinogram-based system matrix to generate a multidimensional image-domain system matrix, which is used for reconstruction of an MPI image from acquired MPI-data. The multi-dimensional sinogram-based system matrix is obtained by performing calibration measurements for N, offsets of the FFL or the FFL trajectory within the calibration volume. In contrast to conventional image-domain calibration, MPI sequence repetitions preferably comprise stepped target positions z, and FFL trajectory offsets (with identical FFL-angles) that are not along the FFL-directions, since it is assumed that the signals received from the spatial positions along the FFL are identical. Thus, the number of calibration measurements can be significantly reduced using this dimensionality reduction. As opposed to conventional image-domain calibrations which require repetitions for each voxel in 3D image-space (calling for a 3-axis robot), sinogram-based calibrations require repetitions for each FFL-offset in the offset dimension of a sinogram and target position z. For example, for a 3D image of size [N_{xy}, N_{xy}, N_{z}], conventional image-domain calibration requires N_{xy}×N_{xy}×N_{z} repetitions, whereas sinogram-based system matrix acquisition requires N_{xy}×N_{z} repetitions (Nᵣ=N_{xy}), while allowing for field based trajectory offsets in e.g. xy-plane.

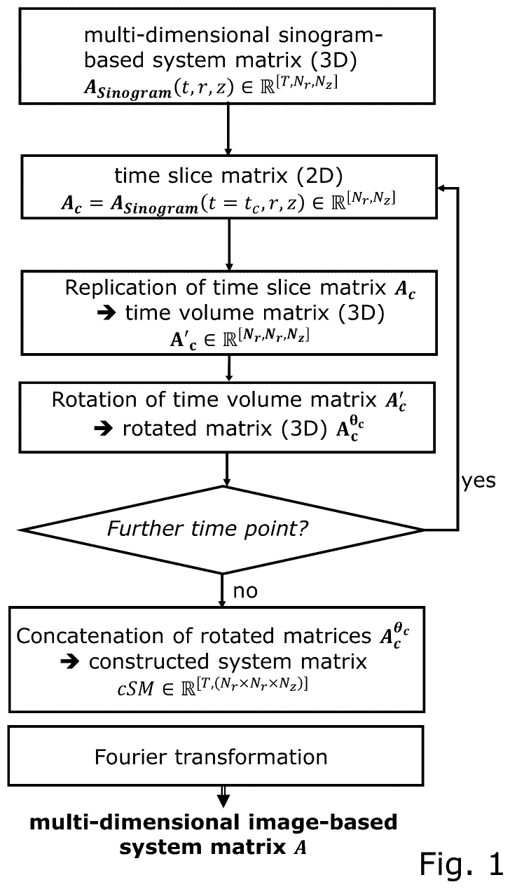

The basic method steps of this method are shown in FIG. 1. In the following, a 3D FOV is assumed (in the case of a 2D FOV, the z-dimension is omitted).

First, time slice matrices A_{c}=A_{Sinogram}(t=t_{c}, r, z)∈ [symbol] ^{[N}^{ᵣ}^{,A}^{_{z}}^{]} are created from the multi-dimensional sinogram-based system matrix for specific time points t_{c} of the trajectory, wherein each time point t_{c} is specific for a time point within the trajectory. Thus, a specific time point t_{c} occurs repeatedly, namely once each time the trajectory is run through. Each time slice matrix A_{c} comprises data of the multi-dimensional sinogram-based system matrix A_{Sinogram} for the specific time point (t=t_{c}). Each time slice matrix A_{c} is replicated Nᵣ−1 times, so that Nᵣ identical time slice matrices A_{c} are obtained (one time slice matrix A_{c} for each FFL-offset). The N identical time slice matrices A_{c} for the specific time point together form a time volume matrix

$${A}_{c}^{′} .$$

The time volume matrix

$${A}_{c}^{′}$$

is then rotated by the FFL angle θ_{c}=θ(t=t_{c}) of the FFL at the specific time point t_{c}, thereby resulting in a rotated matrix

$${A}_{c}^{{θ}_{c}} ,$$

whose dimensions are associated with the spatial dimensions (x, y, z). A constructed system matrix cSM is obtained by concatenating all rotated matrices

$${A}_{c}^{{θ}_{c}}$$

and by vectorising the spatial dimensions after concatenation using the vectorization operator V.

$$cSM = V \{ [ \begin{matrix}{A}_{1}^{{θ}_{1}} \\ {A}_{2}^{{θ}_{2}} \\ ⋮ \\ {A}_{T}^{{θ}_{T}}\end{matrix} ] \} = V \{ [ \begin{matrix}{ℛ}_{{θ}_{1}} \{ Ψ \{ {A}_{1} \} \} \\ {ℛ}_{{θ}_{2}} \{ Ψ \{ {A}_{2} \} \} \\ ⋮ \\ {ℛ}_{{θ}_{T}} \{ Ψ \{ {A}_{T} \} \}\end{matrix} ] \}$$

The multi-dimensional image-domain system matrix A is obtained after performing a Fourier transformation on the constructed system matrix cSM in its time-dimension.

$$A = ℱ \{ cSM \}$$

In the following, three variants are described for obtaining the multi-dimensional sinogram-based system matrix A_{Sinogram}. With all described variants, it is possible to move the calibration source only in one direction within a 3D region of interest ROI (e.g. in z-direction, while staying at fixed position (preferably at the center of the ROI) within the xy-plane) or to not move the calibration source within a 2D region of interest ROI (e.g. at x=0, y=0 within the xy-plane). In order to nevertheless ensure that signals originating from off-centered positions (x≠0|y≠0) are also taken into account in the generation of the system matrix, which is essential for accurate image reconstruction, calibration measurements with corresponding offsets are repeated in accordance with the invention. FIG. 8a shows positions of the calibration source used for calibration with 1D movement of the calibration source, here in the z-direction as example (e.g. for 1D excitation). FIG. 8b and FIG. 8c show offset-positions of the FFL (striped) for the case that an FFL with extension in y-direction for FFL-angle is 0° (FIG. 8b) and x-direction for FFL-angle is 90° (FIG. 8c) is used. The offsets are orthogonal to the extension of the FFL.

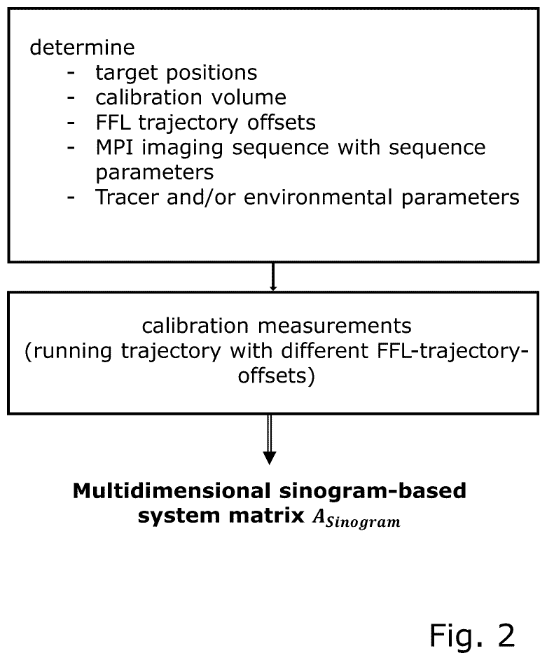

FIG. 2 shows the method steps of a first variant (trajectory offset variant) of generating the multi-dimensional sinogram-based system matrix using a trajectory specific calibration data acquisition by offsetting the trajectory. Calibration measurements are performed in a sinogram domain that is specifically tailored to a given trajectory (e.g. the 3D FFL sinusoidal trajectory shown in FIG. 10a-c).

In contrast to the methods known from the prior art, the first variant of the inventive method does not comprise running a specific trajectory repeatedly for different target positions of the calibration source, but to shift the trajectory by FFL trajectory offsets, wherein for all FFL trajectory offsets, calibration data is acquired during running the trajectory.

First, relevant items required to perform the method are determined, in particular: MPI imaging sequence (trajectory), positions to which the calibration source is to be moved within the region of interest ROI (target positions), calibration volume within which the trajectory is to be offset (size, resolution), FFL trajectory offsets, tracer and environmental parameters, e.g. tracer, temperature of the tracer, viscosity of the tracer environment. The FFL trajectory is preferable not only shifted within the region of interest ROI but also to the overscanning region OS and acquisition window during which a calibration measurement is to be performed.

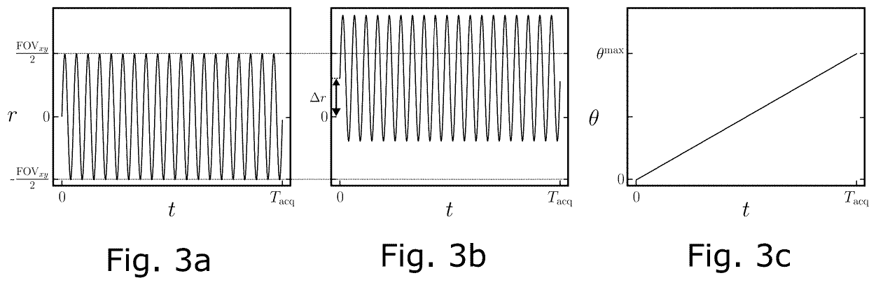

Calibration measurements are performed by running the trajectory, wherein during each calibration measurement the trajectory is run at least once with a specific FFL-trajectory-offset. The FFL-trajectory-offsets are constant during a calibration measurement. The FFL-trajectory-offsets are additional FFL-offsets to the trajectory. Thus, the additional FFL-offset is also always orthogonal to the extension of the FFL, also when the FFL angle changes during the trajectory; i.e. the direction of the additional FFL-offset due to the FFL-trajectory-offset rotates with the rotating FFL. The trajectories with FFL-trajectory-offsets are generated by using additional corresponding alternating (ac) magnetic focus fields. In order to perform the next calibration measurement, a new FFL trajectory offset is set, by using according alternating (ac) focus fields. FIG. 3a,b shows the sinusoidal trajectory shown in FIG. 10 for different FFL trajectory offsets (Δr=0 (FIG. 3a) and Δr=¼FOV (FIG. 3b). The FFL-angle θ does not depend on FFL-trajectory-offset Δr. In other words, additional FFL-trajectory-offset Δr does not change the angle of the FFL for any given time (FIG. 3c).

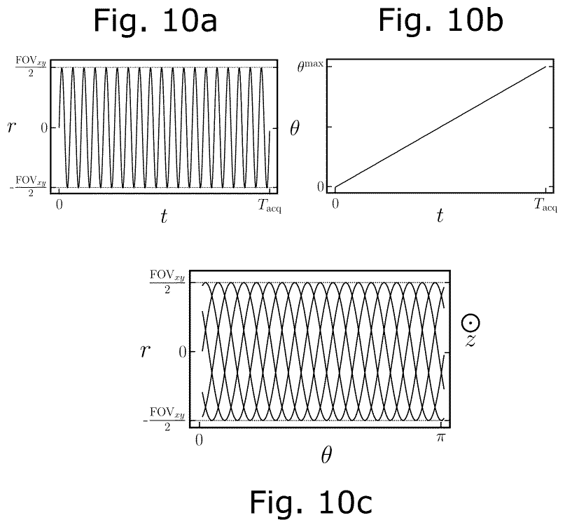

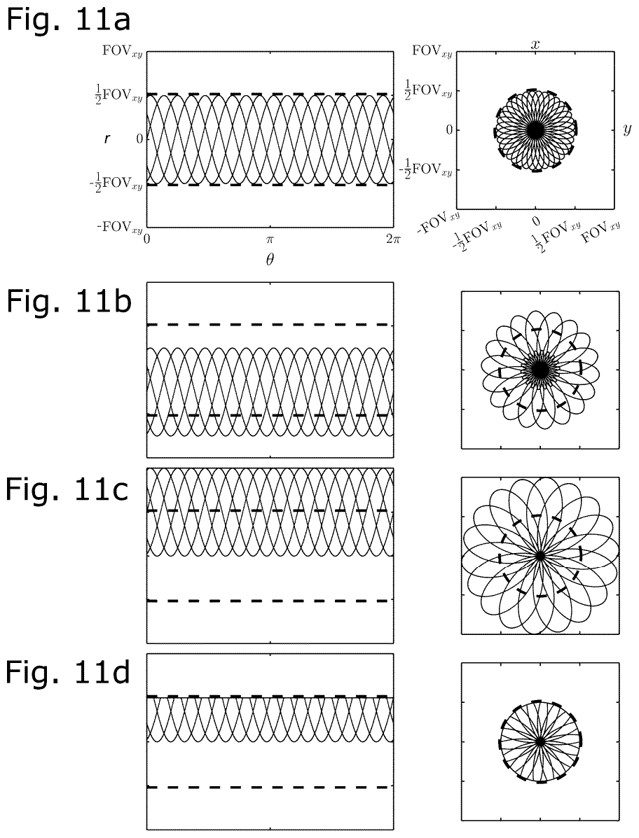

FIG. 11a-d shows a sinusoidal trajectory in sinogram-domain and image-domain with trajectory offset Δr=0 (FIG. 11a), trajectory offset Δr=−¼FOV (FIG. 11b) and trajectory offset Δr=½ FOV (FIG. 11c). The dotted lines represent the boundaries of the FOV, which corresponds to the bore edge of the MPI scanner. Since no data can be acquired outside the FOV, the trajectory can be clipped as shown in FIG. 11d.

By using this sinogram-based calibration data for synthesizing the multidimensional image-domain system matrix in the image domain, the total calibration time is drastically lower than with conventional calibration:

The sinogram-based system calibration sequence for the 3D FFL sinusoidal trajectory shown in FIG. 10 is defined by the following equation:

$${r}_{k} ( t ) = \frac{{FOV}_{xy}}{2} sin ( 2 {π f}_{tra} t ) + {Δ r}_{k}$$

$$where$$

$${Δ r}_{k} = - \frac{{FOV}_{xy}}{2} + k \cdot {d}_{xy} , k ∈ \{ 0 , 1 , … , {N}_{xy} - 1 \} , {d}_{xy} = \frac{{FOV}_{xy}}{{N}_{xy}} ,$$

and N_{xy} are the voxel size and the number of voxels in the xy-directions, respectively. This procedure is repeated for each calibration source position of the tracer probe, preferably in the center of the MPI system bore (x=y=0) along the z-direction:

$${z}_{l} = - \frac{{FOV}_{z}}{2} + l \cdot {d}_{z} , l ∈ \{ 0 , 1 , … , {N}_{z} - 1 \} ,$$

$${d}_{Z} = \frac{{FOV}_{z}}{{N}_{z}}$$

and N_{z} are the voxel size and the number of voxels in the z-direction, respectively. The total measurement time is therefore N_{xy}×N_{z}×T_{acq}, assuming 100% acquisition duty cycle. The resulting sinogram-based calibration data can be represented as Aₛᵢₙₒ(t, r, z)∈ [symbol] ^{T×N}^{_{xy}}^{×N}^{_{z}}, where T is the number of data points per measurement.

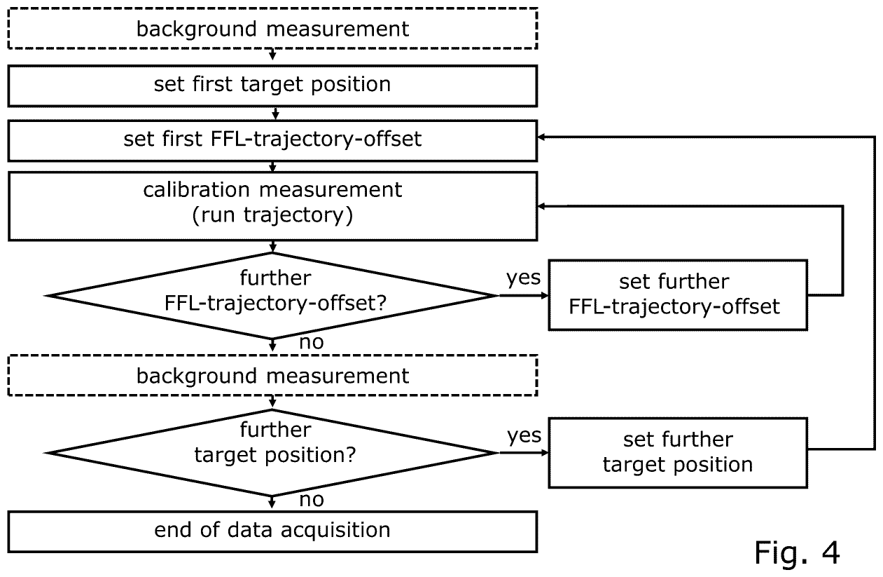

FIG. 4 shows a preferred variant how to perform the calibration measurements. First, the calibration source is positioned in a first target position. Preferably, prior to positioning the calibration source, background measurements can be carried out. The field-free line is positioned at a first FFL-position by setting a first FFL trajectory offset. Starting from this FFL-position, one calibration measurement is performed by running the trajectory. This is repeated for each FFL trajectory offset. Then the calibration source is positioned at a further target position (e.g. by moving the calibration source mechanically) and the whole procedure is repeated for all target positions. Before setting each new target position, an optional background measurement is performed.

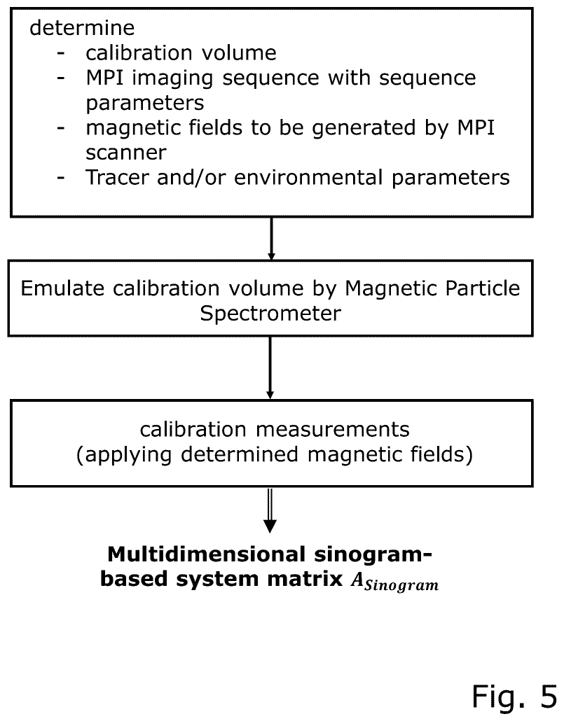

FIG. 5 shows the method steps of a second variant (MPS variant) of generating the multi-dimensional sinogram-based system matrix using a Magnetic Particle Spectrometer for emulating the calibration volume.

In contrast to the methods known from the prior art, the second variant of the inventive method does not use 3D fields with gradients generated by an MPI scanner but a homogeneous field corresponding to a specific voxel position of MPI scanner at each time point generated by the Magnetic Particle Spectrometer. In this case, there is no magnetic field gradient and therefore no field free region.

First, relevant items required to perform the method are determined, in particular: Calibration volume of an MPI scanner in sinogram domain, from which calibration data is to be acquired, MPI scanner parameters (including drive field frequency, drive field amplitude, and magnetic field gradient G of an MPI scanner), MPI imaging sequence (trajectory), tracer and/or environmental parameters (e.g., temperature and viscosity), magnetic fields B(t, Δr, z) to be generated by an MPI scanner in order to emulate the magnetic fields of an FFL trajectory for only one voxel position (for each target position) of an MPI scanner. The calculated fields depend on the MPI scanner parameters and the trajectory. In other words: The magnetic fields that an MPI scanner would generate when running the determined MPI sequence are calculated. These calculated magnetic fields are then applied to the calibration source in the MPS.

The trajectory is preferably not only shifted within the region of interest ROI but also to the overscanning region OS and acquisition window during which a calibration measurement is to be performed.

Instead of moving the calibration source mechanically, the varying z-positions of the calibration source is emulated by applying additional constant (dc) fields in the z-direction using the Magnetic Particle Spectrometer.

By acquiring sinogram-based calibration data using MPS for synthesizing the multidimensional system matrix in the image domain, the MPI scanner is not blocked during calibration measurements, reducing the downtime of the imaging device. Moreover, signals acquired with MPS can have significantly higher SNR, allowing generation of system matrices with higher spatial resolution and more accuracy.

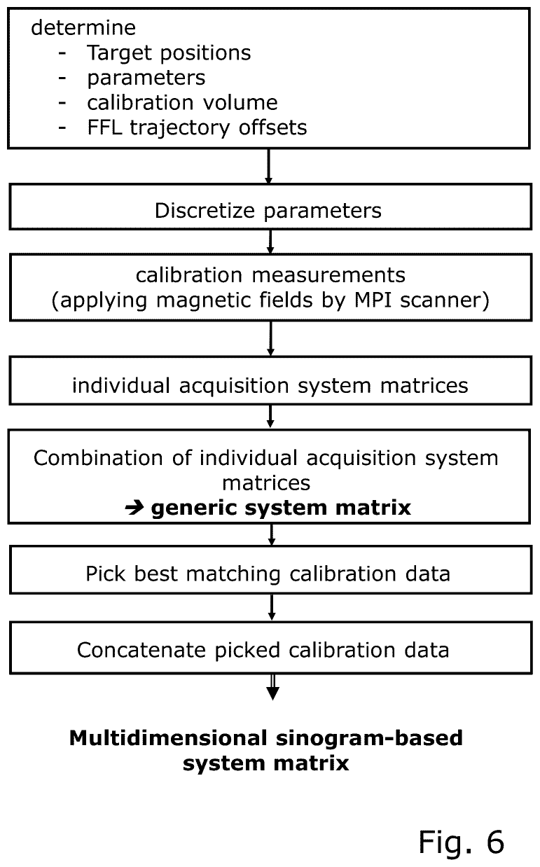

FIG. 6 shows the method steps of a third variant (generic variant) of generating the multi-dimensional sinogram-based system matrix using a generic calibration data acquisition method independent of a specific trajectory.

In contrast to the methods known from the prior art, the third variant of the inventive method does not comprise running a specific trajectory repeatedly for different target positions of the calibration source, but to shift the FFL and acquire data with various parameter value combinations for the respective FFL position, wherein calibration data is acquired for all FFL-offsets and all set parameter value combinations.

First, relevant items required to perform the method are determined, in particular: positions to which the calibration source is to be moved within the region of interest ROI (target positions), calibration voxels from which signals are to be acquired (calibration voxels), parameters to be varied in order to get information for different parameter value combinations, positions to which the FFL is to be shifted by setting respective FFL-offsets. The FFL is preferable not only shifted within the region of interest ROI but also to the overscanning region OS and acquisition window during which a calibration measurement is to be performed.

In contrast to running complete trajectories (as e.g. in variant 1), the parameters are discretized before the calibration measurements are performed. After the calibration measurements, individual acquisition system matrices (with dimensions frequency (or time) and FFR-offset) are determined. By combining the individual system matrices A_{c} the multidimensional system matrix A is acquired. For example, for the multidimensional system matrix A with dimensions frequency, FFL-offset, calibration source positions z along the z-direction at the center of the ROI (x=y=0), FFL velocity, and FFL angle, can be represented in terms of is individual acquisition system matrices A_{c} as following:

$$A ( f , r , z , v = {v}_{c} , θ = {θ}_{c} ) = {A}_{c} ( f , r , z )$$

where the individual system matrices A_{c} is acquired for FFL velocity v_{c} and FFL angle θ_{c}.

From the generic system matrix the best matching calibration data (i.e. calibration data of that calibration measurement that best matches to the combination of values of the predetermined parameters of the respective drive field cycle of an MPI measurement from which an MPI image is to be reconstructed) is picked and concatenated, thereby obtaining the multi-dimensional sinogram-based system matrix.

In contrast to the methods known from the prior art, the third variant of the inventive method does not comprise running a specific trajectory repeatedly for different target positions of the calibration source, but provides for shifting the FFL to predefined calibration voxels and setting a multitude of parameter value combinations, wherein a calibration data is acquired for all FFL-offsets and all set parameter value combinations.

### LIST OF REFERENCE SIGNS

CV calibration volume
ROI region of interest
OS overscanning region
T trajectory
FFL field free line

1. A method of measuring a region of interest (ROI) with a magnetic particle imaging (MPI) measurement system, the method comprising:
providing an MPI scanner using an MPI-sequence with a field free line (FFL) with FFL angles θ, FFL offsets r, and FFL velocities;
performing calibration measurements and acquiring a multi-dimensional sinogram-based system matrix A_{Sinogram}(t, r, z)∈ [symbol] ^{[T,N}^{ᵣ}^{,N}^{_{z}}^{]}, from the calibration measurements, the sinogram having an offset-dimension, an angle-dimension, and a z-dimension, wherein
$$r = FFL - offset in the offset - dimension of the sinogram$$
$$θ = angle in the angle - dimension of the sinogram$$
$$t = time in the time - dimension of the multi - dimensional sinogram - based system matrix {A}_{Sinogram} \text{} and \text{} multi - dimensional image - domain system matrix A$$
$${N}_{r} = number of FFL - offsets$$
$${N}_{z} = number of voxels of the ROI in a z - direction$$
$$T = number of time points of the time dimension ( number of calibration data points during an MPI imaging sequence ) ;$$
for each time point t_{c}, c∈[1, T]:
getting a time slice matrix A_{c}=A_{Sinogram}(t=t_{c}, r, z)∈ [symbol] ^{[N}^{ᵣ}^{,N}^{_{z}}^{]}, which comprises calibration data of the multi-dimensional sinogram-based system matrix A_{Sinogram}(t, r, z) at time point t=t_{c}, the time slice matrix having a 1^{st} offset-dimension r and a z-dimension z,
replicating the time slice matrix A_{c} Nᵣ−1 times, resulting in Nᵣ identical time slice matrices A_{c}, and copying these replications along a newly added 2^{nd} offset-dimension, resulting in a time volume matrix
$${A}_{c}^{′} ∈ {ℝ}^{[ {N}_{r} , {N}_{r} , {N}_{z} ]} ,$$
and
rotating the time volume matrix A_{c}′ by FFL angle θ_{c}=0 (t=t_{c}) of a respective field free line (FFL) at time point t=t_{c} in its 1^{st} and 2^{nd} offset-dimensions, resulting in a rotated matrix
$${A}_{c}^{{θ}_{c}} ,$$
where dimensions of
$${A}_{c}^{{θ}_{c}}$$
are associated with spatial dimensions (x, y, z);
concentrating the rotated matrices
$${A}_{c}^{{θ}_{c}}$$
of all time points along a time-dimension and vectorizing the spatial dimensions (x, y, z), resulting in a constructed system matrix cSM∈ [symbol] ^{[T,(N}^{ᵣ}^{×N}^{ᵣ}^{×N}^{_{z}}^{)]};
performing a Fourier transformation on the constructed system matrix cSM on its time-dimension to generate a multi-dimensional image-domain system matrix A; and
performing MPI measurements of the ROI with the MPI scanner, using the system matrix A to determine signal contributions of voxels within the ROI.

2. The method according to claim 1, wherein the multi-dimensional sinogram-based system matrix A_{Sinogram}(t, r, z)∈ [symbol] ^{[T,N}^{ᵣ}^{,N}^{_{z}}^{]} is generated according to the following method:
prior to calibration measurements, determining:
target positions for a calibration source,
a calibration volume (CV) in sinogram domain from which calibration data is to be acquired, the calibration volume (CV) comprising the region of interest (ROI),
MPI imaging sequence with a trajectory (TRA) comprising sequence parameters, the sequence parameters including FFL angular frequency, FFL translational frequency, and MPI imaging sequence duration,
tracer and/or environmental parameters, and
FFL-trajectory-offsets Δr, by which the positions of the field free line (FFL) during the trajectory (TRA) are to be shifted within the calibration volume (CV) perpendicular to the respective orientation of the field free line (FFL);
performing calibration measurements by running the MPI imaging sequence at least once during each calibration measurement with a specific FFL-trajectory offset and a specific target position z using the MPI scanner, wherein the calibration measurements differ in the combination of FFL-trajectory-offset Δr and target position z; and
determining the multi-dimensional sinogram-based system matrix A_{Sinogram} from the calibration data.

3. The method according to claim 2, wherein performing the calibration measurements comprises the following steps:
i) positioning the calibration source within the calibration volume (CV) at a first target position;
ii) acquiring calibration data with the calibration source being positioned at a first target position, wherein the trajectory (TRA) of the MPI sequence is run through until all FFL-trajectory-offsets Δr have been set, wherein the FFL-trajectory-offset Δr is changed after the trajectory (TRA) is run through;
iii) positioning the calibration source within the region of interest (ROI) at a further target position that is shifted relative to the previous target position within the calibration volume (CV) in at least one direction;
iv) running through the trajectory (TRA) of the MPI sequence until all FFL-trajectory-offsets Δr have been set, wherein the FFL-trajectory-offset Δr is changed after the trajectory (TRA) is run through with the calibration source being positioned at the further target position; and
v) repeating steps (iii)-(iv) until measurements for all target positions of the calibration source are performed.

4. The method according to claim 3, further comprising performing background measurements prior to positioning the calibration source at the target positions in step (i) and step (iii), without the calibration source being in the calibration volume (CV) and/or with the calibration source being in a magnetically saturated state.

5. The method according to claim 1, wherein the multi-dimensional sinogram-based system matrix A_{Sinogram}(t, r, z)∈ [symbol] ^{[T,N}^{ᵣ}^{,N}^{_{z}}^{]} is generated according to the following method using a magnetic particle spectrometer for emulating a calibration volume (CV), the method comprising:
prior to calibration measurements, determining
calibration volume (CV) in sinogram domain, the calibration volume (CV) from which calibration data is to be acquired, the calibration volume (CV) comprising the region of interest (ROI),
MPI scanner parameters of the MPI scanner including drive field frequency, drive field amplitude, and magnetic field gradient G,
MPI imaging sequence with a trajectory (TRA) comprising sequence parameters, the sequence parameters including FFL angular frequency, FFL translational frequency, and MPI imaging sequence duration,
tracer and/or environmental parameters,
FFL-trajectory-offsets Δr,
spatial positions z of the calibration volume (CV), and
for each combination of FFL-trajectory-offset Δr and spatial position z, time dependent magnetic fields B(t, Δr, z), wherein each magnetic field B(t, Δr, z) corresponds to the magnetic field generated by the MPI scanner during a trajectory run with a specific FFL trajectory offset Δr at a specific spatial position z;
performing calibration measurements by emulating the calibration volume (CV) by applying the determined magnetic fields B(t, Δr, z) to a calibration source, wherein for each combination of FFL-trajectory-offset Δr and spatial position z a calibration measurement is performed during which the corresponding magnetic field B(t, Δr, z) is applied at least once, and calibration data are acquired; and
determining the multi-dimensional sinogram-based system matrix A_{Sinogram} from the calibration data.

6. The method according to claim 1, wherein the multi-dimensional sinogram-based system matrix A_{Sinogram}(t, r, z)∈ [symbol] ^{[T,N}^{ᵣ}^{,N}^{_{z}}^{]} is generated according to the following method, wherein the offset is an FFL-offset:
performing generic MPI calibration measurements by applying at least one drive field cycle of a drive field in each calibration measurement using an MPI scanner, wherein during each generic MPI calibration measurement data is acquired for a specific combination of values of target position for a calibration source, FFL offset, and of predetermined parameters, comprising FFL angle and FFL velocities; and
determining a generic system matrix A_{generic}, where A_{generic} comprises calibration data acquired from the generic MPI calibration measurements;
and wherein trajectory-specific calibration data is selected from the generic system matrix A_{generic} according to the following method:
for each drive field cycle of an MPI measurement,
determining the FFL angle, FFL offset, and FFL velocity, and
picking the calibration data from the generic system matrix A_{generic} of that calibration measurement that best matches to the combination of values of the predetermined parameters of the respective drive field cycle; and
concatenating all picked calibration data along the time dimension, resulting in the multi-dimensional sinogram-based system matrix A_{Sinogram}.

7. The method according to claim 6, wherein the determined parameters comprise at least one of: particle type of used tracer of the calibration source, temperature of the calibration source, viscosity of the calibration source, velocity of the calibration source, drive field frequency, drive field amplitude, magnetic field gradient of an MPI system used for the MPI measurements, duration of the trajectory (TRA), position of optional hardware inserts, shape of the field free line (FFL), receiver coils used, and receiver coil orientations.

8. The method according to claim 1, wherein for setting a specific FFL-trajectory-offset Δr of the MPI sequence or for setting a specific FFL offset Δr, the field free line (FFL) is shifted in the offset-dimension of the sinogram by applying additional focus fields.

9. The method according to claim 1, wherein multi-dimensional sinogram-based system matrix A_{Sinogram} is clipped in at least one spatial dimensions (r, z) of the multi-dimensional sinogram-based system matrix A_{Sinogram}, such that the multi-dimensional sinogram-based system matrix A_{Sinogram}, only comprises calibration data from field offsets lower than a threshold rₗᵢₘ, zₗᵢₘ.

10. The method according to claim 1, wherein during each calibration measurement, averaging is applied, and the number of averages is set for a targeted signal-to-noise-ratio SNR.

11. The method according to claim 1 wherein, in order to move the calibration source between the target positions, the calibration source is moved relative to the region of interest (ROI) exclusively in the drive field direction.

12. The method according to claim 1, wherein a fully-sampled calibration data consists of n measurements, the number of calibration measurements m, is smaller than the number n, and calibration data not measured is estimated, in particular by interpolation and/or extrapolation using already acquired calibration data.

13. The method according to claim 1, wherein image reconstruction is performed using multi-dimensional image-domain system matrix A, by providing an MPI measurement b and calculating a concentration of particles with A and b.

14. The method according to claim 13, wherein an iterative reconstruction algorithm is used for calculating the concentration of the particles, based on the following forward model:
$${argmin}_{ρ} {‖ A ρ - b ‖}_{2}^{2} + λ Φ ( ρ )$$
$$where$$
$$A = multi - dimensional image - domain system matrix ,$$
$$ρ = MPI - image$$
$$b = measurement vector ,$$
$$λ = regularization constant for regularization operation , and$$
$$Φ ( \cdot ) = regularization operator .$$
